(19) United States
(12) Patent Application Publication
(10) Pub. No.: US 2020/0306509 A1
(43) Pub. Date: Oct. 1, 2020

(54) GUIDEWIRE AND METHOD THEREFOR

(71) Applicant: Lake Region Medical, Inc., Wilmington, MA (US)

(72) Inventors: Harshad Holehonnur, Galway (IE); Joe Freeley, Galway (IE)

(21) Appl. No.: 16/823,409

(22) Filed: Mar. 19, 2020

Related U.S. Application Data
(60) Provisional application No. 62/825,048, filed on Mar. 28, 2019.

(51) Int. Cl. A61M 25/09; A61M 25/00

(52) U.S. Cl. CPC A61M 25/09; A61M 25/001; A61M 25/0054; A61M 2025/09133; A61M 2025/09108; A61M 2025/09091; A61M 25/0012

(57) ABSTRACT

In various examples, a method of making a guidewire is described. The method includes placing a polymer jacket over a core wire. The core wire includes a first portion having a first profile and a second portion having a second profile. The first profile is smaller than the second profile. Heat is applied to the polymer jacket and the core wire to reflow the polymer jacket and fuse the polymer jacket to the core wire, wherein the polymer jacket forms a layer of substantially uniform thickness along a length of the core wire. In some examples, a guidewire made using the method is also described.

### CROSS REFERENCE TO RELATED APPLICATIONS

This application claims the benefit of priority to U.S. Provisional Application Ser. No. 62/825,048, filed on Mar. 28, 2019, entitled “GUIDEWIRE WITH POLYMER FOLLOWING THE SHAPE OF DISTALLY TAPERED CORE WIRE,” which is incorporated by reference herein in its entirety.

### BACKGROUND

Navigating through the vasculature of a patient can be challenging, especially as the vasculature becomes narrow, tortuous, or built up with plaque, for instance. Navigating conventional guidewires through such vasculature can become problematic due to distal ends of the conventional guidewires being too large to make it through tight bends, small channels or openings, or the like. Difficulty in navigating through vasculature and/or accessing locations within a patient can delay and/or complicate procedures, increasing the procedure time, the expense, and the risk of complications to the patient.

### OVERVIEW

This overview is intended to provide an overview of subject matter of the present patent document. It is not intended to provide an exclusive or exhaustive explanation of the invention. The detailed description is included to provide further information about the present patent document.

The present inventors have recognized, among other things, that the present subject matter can be used to provide a guidewire with a relatively small distal tip profile to allow for the guidewire to be maneuvered through smaller vasculature, channels, openings, or the like than conventional guidewires. In various examples, the present subject matter is advantageous in that it provides for a guidewire that includes a more flexible distal tip than conventional guidewires to enable, for instance, increased free movement of the distal tip while maintaining longitudinal stiffness and force response of the distal tip. In some examples, the present invention facilitates access to branched vasculature with an acute or otherwise difficult-to-navigate bend. In some examples, the present invention can facilitate the guidewire in crossing/penetrating a lesion with a reduced risk of rupture. To better illustrate the devices and methods described herein, a non-limiting list of examples is provided here:

Example 1 can include subject matter that can include a method of making a guidewire. The method includes placing a polymer jacket over a core wire. The core wire includes a first portion having a first thickness and a second portion having a second thickness. The first thickness is smaller than the second thickness. Heat is applied to the polymer jacket and the core wire to reflow the polymer jacket and fuse the polymer jacket to the core wire, wherein the polymer jacket forms a layer of substantially uniform thickness along a length of the core wire.

In Example 2, the subject matter of Example 1 is optionally configured such that applying the heat to the polymer jacket and the core wire includes fusing the polymer jacket to the core wire such that a first profile of the polymer jacket and the core wire at the first portion of the core wire is smaller than a second profile of the polymer jacket and the core wire at the second portion of the core wire.

In Example 3, the subject matter of Example 1 or 2 is optionally configured such that placing the polymer jacket over the core wire includes the first portion of the core wire being proximate a distal end of the core wire.

In Example 4, the subject matter of any one of Examples 1-3 is optionally configured such that placing the polymer jacket over the core wire includes the second portion of the core wire being disposed between the first portion and a proximal end of the core wire.

In Example 5, the subject matter of any one of Examples 1-4 optionally includes placing heat shrink tubing over the polymer jacket prior to applying heat to the polymer jacket and the core wire, wherein the heat shrink tubing constrains the polymer jacket with application of the heat to the polymer jacket and the core wire. The heat shrink tubing is removed from the polymer jacket and the core wire after applying the heat to the polymer jacket and the core wire.

In Example 6, the subject matter of any one of Examples 1-5 is optionally configured such that placing the polymer jacket over the core wire includes the core wire having a tapered portion between the first portion and the second portion.

In Example 7, the subject matter of any one of Examples 1-6 optionally includes placing a coil over the core wire, wherein the coil is disposed completely within the polymer jacket after the polymer jacket is reflowed.

In Example 8, the subject matter of Example 7 is optionally configured such that placing the coil over the core wire includes placing the coil over the first portion of the core wire.

In Example 9, the subject matter of any one of Examples 1-8 is optionally configured such that applying heat to the polymer jacket and the core wire includes reflowing the polymer jacket to form the layer of substantially uniform thickness along the first portion of the core wire.

In Example 10, the subject matter of Example 9 is optionally configured such that applying heat to the polymer jacket and the core wire includes achieving the layer of substantially uniform thickness along the first portion of the core wire without trimming down the polymer jacket.

Example 11 can include, or can optionally be combined with any one of Examples 1-10 to include subject matter that can include a guidewire. The guidewire includes a core wire including a first portion having a first thickness and a second portion having a second thickness. The first thickness is smaller than the second thickness. A polymer jacket is reflowed over the core wire to fuse the polymer jacket to the core wire, wherein a layer of the polymer jacket includes a substantially uniform thickness along a length of the core wire.

In Example 12, the subject matter of Example 11 is optionally configured such that the first portion of the core wire is proximate a distal end of the core wire.

In Example 13, the subject matter of Example 11 or 12 is optionally configured such that the second portion of the core wire is disposed between the first portion and a proximal end of the core wire.

In Example 14, the subject matter of any one of Examples 11-13 is optionally configured such that a first profile of the polymer jacket and the core wire at the first portion of the core wire is smaller than a second profile of the polymer jacket and the core wire at the second portion of the core wire.

In Example 15, the subject matter of any one of Examples 11-14 is optionally configured such that the core wire includes a tapered portion between the first portion and the second portion.

In Example 16, the subject matter of any one of Examples 11-15 optionally includes a coil disposed over the core wire, the coil being disposed completely within the polymer jacket after the polymer jacket is reflowed.

In Example 17, the subject matter of Example 16 is optionally configured such that the coil is disposed over the first portion of the core wire.

In Example 18, the subject matter of any one of Examples 11-17 is optionally configured such that the polymer jacket is reflowed to form the layer of substantially uniform thickness along the first portion of the core wire.

In Example 19, the subject matter of any one of Examples 11-18 is optionally configured such that the first profile of the guidewire includes a thickness of less than 0.014 inches.

In Example 20, the subject matter of any one of Examples 11-19 is optionally configured such that the first profile of the guidewire includes a thickness of about 0.01 inches.

### BRIEF DESCRIPTION OF THE DRAWINGS

FIG. 1 is a cross-sectional view of a guidewire in accordance with at least one example of the invention.

FIG. 2 is a cross-sectional view of a guidewire made by trimming a polymer jacket to size.

FIG. 3 is a perspective view of a branched artery with plaque build-up through which a guidewire in accordance with at least one example of the invention can be passed.

FIG. 4 is a partial cross-sectional view of a branched artery for which a guidewire in accordance with at least one example of the invention can be used.

FIGS. 5A-5E depict a method of making the guidewire of FIG. 1, the method in accordance with at least one example of the invention.

FIGS. 6A-6F depict a method of making a guidewire of FIG. 2.

### DETAILED DESCRIPTION

The present invention relates generally to providing a guidewire with a relatively small distal tip profile to allow for the guidewire to be maneuvered through smaller vasculature, channels, openings, or the like than conventional guidewires. More specifically, the present invention relates to a method of making a guidewire that includes a more flexible distal tip than conventional guidewires to enable, for instance, increased free movement of the distal tip while maintaining longitudinal stiffness and force response of the distal tip. In some examples, the present invention facilitates access to branched vasculature with an acute or otherwise difficult-to-navigate bend. In some examples, the present invention can facilitate the guidewire in crossing/penetrating a lesion with a reduced risk of rupture.

Tip shaping of a conventional guidewire is a way to get access to a branch of artery including an acute bend. This can be done by shaping the tip of the conventional guidewire in the same direction as the acute bend of the artery, either during manufacture (J tip) or before the medical procedure (by the physician). However, tip shaping can be done only be in one direction. The shaping will not be effective in a case of acute multiple bends in opposite or different directions. Manual shaping may reduce the torque response as well. In some examples, the guidewire of the present subject matter results in a guidewire which can be flexible in multiple directions. The guidewire of the present subject matter can also navigate multiple bends without significantly affecting the torque response.

A flexible coil can be used at a distal end of a conventional guidewire to gain access to an acute bend. The flexible coil of the conventional guidewire is attached to the core wire at a joint. Due to the joint, the torque response is lower between the proximal end and the distal end of the conventional guidewire. In the guidewire of the present subject matter, a polymer jacket is completely attached to the core to give improved force/torque transfer between proximal and distal ends of the guidewire. Chemical bonds formed between hydrophilic coating and polymer are also stronger than that of hydrophilic coating and metallic coils.

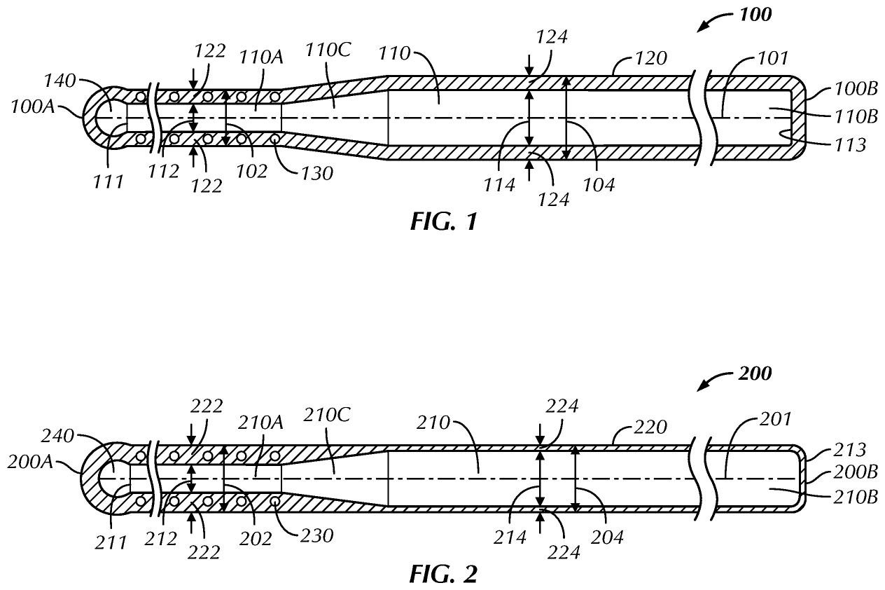

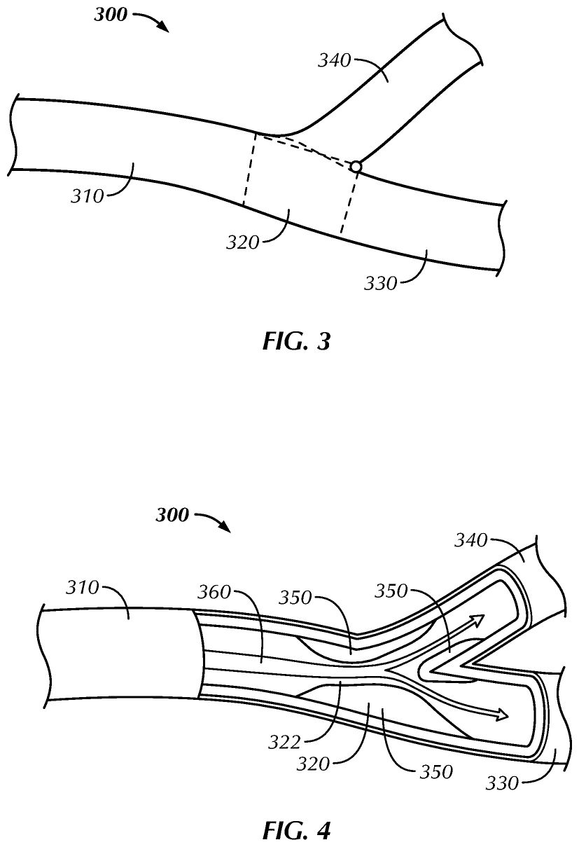

In some examples, referring briefly to FIG. 1, the present subject matter can be used to increase the free movement of a distal portion 100A of a guidewire 100 because a polymer jacket 120 closely follows a shape of a core wire 110 underneath, making a distal portion 100A of the guidewire 100 more flexible. When compared to a conventional guidewire, such as a guidewire 200 seen in FIG. 2, a first profile 102 of the distal portion 100A of the guidewire 100 of FIG. 1 is noticeably smaller than a first profile 202 of a distal portion 200A of the conventional guidewire 200, leading to more flexibility in the guidewire 100 of the present subject matter as compared to the conventional guidewire 200. Such increased flexibility of the distal portion 100A of the guidewire 100, in some examples, can provide easier access to a branch of an artery that has an acute bend to maneuver without prolapse. For instance, referring to FIG. 3, the present subject matter can facilitate access to a side branch 340 of a branched artery 300. In some examples, lowering the first profile 102 of the guidewire 100 can improve the access to a calcified vessel with a microchannel 322, as seen in FIG. 4. In some examples, the present subject matter can facilitate the guidewire 100 in crossing/penetrating a lesion 350 (such as, for instance, a buildup of plaque 350) with reduced risk of rupture of the artery 300.

Referring now to FIGS. 3 and 4, the example of the artery 300 shown includes a proximal segment 310 leading to a bifurcation core 320 where the artery 300 splits between a distal segment 330 and the side branch 340. In some examples, the artery 300 can include the lesion 350, such as the buildup of plaque 350. In some instances, access to the side branch 340 of the artery 300 can be difficult due to the buildup of plaque 350. In some examples, the present subject matter facilitates navigation through the microchannel 322 within the buildup of plaque 350 in order to cross or penetrate the lesion 350. In some examples, the buildup of plaque 350 can result in reduced blood flow 360 through the artery 300, which can decrease the amount of oxygen reaching organs and/or other parts of a patient. Over time, the buildup of plaque 350 can lead to serious health issues for the patient, including stroke, heart attack, and/or death, to name a few.

Although the present subject matter is described with respect to accessing the side branch 340 of the artery 300, it should be understood that the present subject matter can be used to access other vessels with geometries different from that shown in FIGS. 3 and 4. Moreover, while the present subject matter is believed to be advantageous with respect to crossing or penetrating the buildup of plaque 350, it should be understood that the examples of the present subject matter can be used in vessels with or without plaque 350 or other lesions.

Referring to FIG. 2, the conventional guidewire 200 includes a generally uniform profile along a length of the guidewire 200. That is, the first profile 202 of the distal portion 200A of the guidewire 200 is substantially similar in size to a second profile 204 of a proximal portion 200B of the guidewire 200. As is described in more detail below, this is due to the manner in which the guidewire 200 is manufactured. The guidewire 200 of FIG. 2 includes a core wire 210 disposed within a polymer jacket 220 having a distal end 211 and a proximal end 213. The core wire 210 extends along a longitudinal axis 201 of the guidewire 200 and includes a distal portion 210A having a distal diameter or thickness 212 and a proximal portion 210B having a proximal diameter or thickness 214, the distal diameter or thickness 212 being smaller than the proximal diameter or thickness 214. However, the polymer jacket 220 includes a distal wall thickness 222 that is greater than a proximal wall thickness 224, such that the overall profile of the guidewire is substantially uniform along the length of the guidewire 200. The core wire 210 of the guidewire 200 can include a tapered portion 210C to transition from the smaller distal portion 210A to the larger proximal portion 210B of the core wire 210. The guidewire 200 of FIG. 2 shows a coil 230 disposed at the distal portion 200A of the guidewire 200 and completely retained within the polymer jacket 220, such that the coil 230 is disposed completely beneath a surface of the polymer jacket 220. The guidewire 200 can include a tip 240 disposed at the distal end 211 of the core wire 110. The tip 240 can be rounded to facilitate atraumatic insertion of the guidewire 200. With such a configuration, the conventional guidewire 200, while capable of navigating through some vasculature, can run into problems navigating smaller vessels, channels, microchannels, or the like due to the relatively large first profile 202 of the distal portion 200A of the guidewire 200. What is needed is an improved guidewire including a reduced distal profile to facilitate navigation through smaller vessels, channels, microchannels, and the like and an improved method of making such a guidewire.

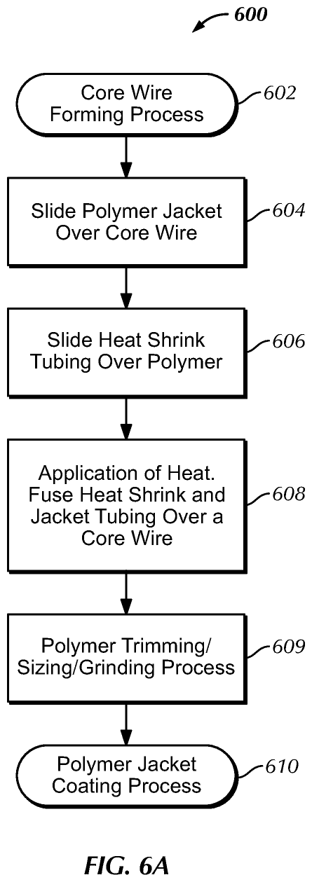

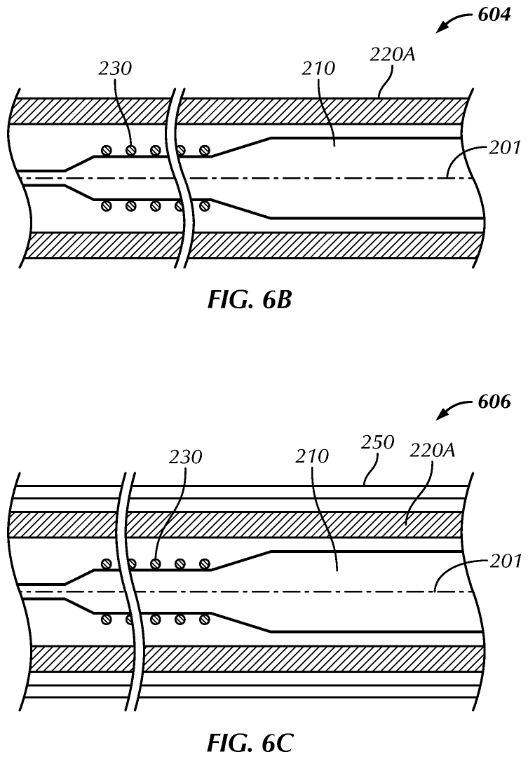

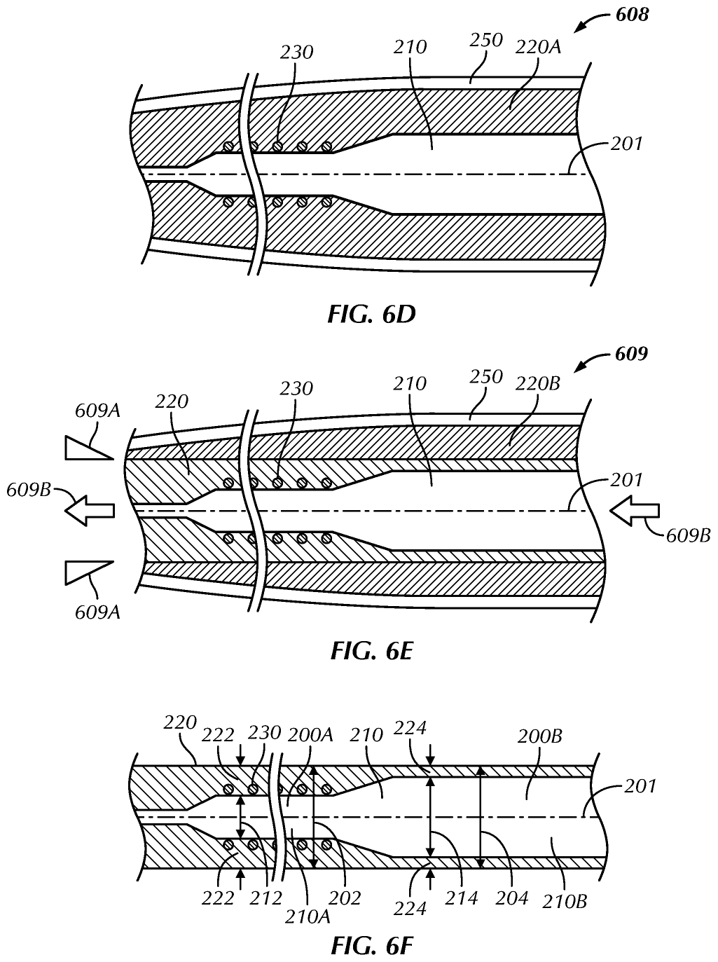

Referring now to FIGS. 6A-6F, a method 600 for manufacturing the guidewire 200 is shown. Currently, manufacturing of the guidewire 200 involves attaching the polymer jacket 220 to the core wire 210 followed by trimming down of the polymer jacket 220 to size the polymer jacket 220. Initially, formation 602 of the core wire 210 is performed to obtain the core wire 210 with the desired dimensions and characteristics. Once the core wire 210 is formed, the polymer jacket 220A is slid, placed, or otherwise disposed 604 over the core wire 210. Optionally, the coil 230 is also placed at least partially around the core wire 210 and under the polymer jacket 220A. Once the desired components are configured and placed, heat shrink tubing 250 is placed 606 over the polymer jacket 220A. Heat is then applied 608 to fuse the heat shrink tubing 250 and the polymer jacket 220A over the core wire 210. After heat shrinking, the polymer jacket 220 includes a larger outer diameter than is desired. For this reason the polymer jacket 220A is then trimmed 609 to size. The polymer jacket 220A undergoes the sizing process 609 to achieve a smaller outer diameter. The heat shrink tubing 250 is also removed at this time. The polymer jacket 220A is ground down, trimmed, cut, or otherwise sizeded using one or more cutting tools 609A. However, this sizing process 609 results in a generally constant outer diameter of the polymer jacket 220 along the length of the guidewire 200. The one or more cutting tools 609A are set at a desired distance from the longitudinal axis 201 of the guidewire 200, and the polymer jacket 220A and the heat shrink tubing 250 fused to the core wire 210 are passed in a direction 609B along the longitudinal axis 201 of the guidewire 200 through or by the one or more cutting tools 609A, such that the one or more cutting tools 609A act to remove an excess polymer jacket 220B and the heat shrink tubing 250 to leave the polymer jacket 220. After sizing or trimming 609 of the polymer jacket 220, the polymer jacket 220 can then be coated 610. Various coatings are contemplated for the coating process 610, including, but not limited to a hydrophilic coating, a hydrophobic coating, a coating with at least some radiopacity, or the like.

The sizes of the first profile 202 and the second profile 204 of the guidewire 200 are determined by the distance at which the one or more cutting tools 609A are set from the longitudinal axis 201 of the guidewire 200. However, because the distance of the one or more cutting tools 609A from the longitudinal axis 201 remains fixed, the size of the first profile 202 of the distal portion 200A of the guidewire 200 is substantially the same as the size of the second portion 204 of the proximal portion 200B of the guidewire 200. That is, the sizing process 609 results in a straight profile along the length of the guidewire 200. Because of this, and because the distal diameter or thickness 212 of the distal portion 210A of the core wire 210 is smaller than the proximal diameter or thickness 214 of the proximal portion 210B of the core wire 210, the distal wall thickness 222 is thicker than the proximal wall thickness 224. In this way, even though the distal portion 210A of the core wire 210 is relatively thin, the increased distal wall thickness 222 counteracts any size advantages to the smaller distal portion 210A of the core wire 210, such as the ability to fit into and access smaller channels, vessels, etc. Moreover, the increased distal wall thickness 222 of the polymer jacket 220 decreases flexibility of the distal portion 200A of the guidewire, also limiting the capabilities of the guidewire 200 to navigate into and otherwise access smaller channels, vessels, etc.

Referring now to FIG. 1, the guidewire 100 of the present invention allows for the first profile 102 of the distal portion 100A of the guidewire 100 to be considerably smaller than the first profile 202 of the distal portion 200A of the conventional guidewire 200. In some examples, the guidewire 100 includes the core wire 110 extending along a longitudinal axis 101 of the guidewire 100 and including a distal portion 110A having a distal diameter or thickness 112 and a proximal portion 110B having a proximal diameter or thickness 114, the distal diameter or thickness 112 being smaller than the proximal diameter or thickness 114. It should be understood that, although described in terms of having a diameter or thickness, the core wire 110 need not include a generally circular cross section. The guidewire 100, in various examples, can include various cross-sectional shapes, including, but not limited to, square-shaped, triangular, rectangular, flattened, ovular, elliptical, polygonal, star-shaped, or the like. The core wire 110, in some examples, includes a distal end 111 and a proximal end 113. The distal portion 110A of the core wire 110 is disposed proximate the distal end 111, and the proximal portion 110B is disposed between the distal portion 110A and the proximal end 113 of the core wire 110. In some examples, the proximal portion 110B is disposed proximate the proximal end 113 of the core wire 110. In some examples, the core wire 110 can be formed from one or more metal materials, such as stainless steel, aluminum, MP35N, nickel, titanium, copper, gold, or the like. In further examples, the core wire 110 can be formed from shape memory alloys, such as Nitinol or other combinations of two or more of nickel, titanium, copper, aluminum, gold, or the like. In some examples, the core wire 110 can be formed from a polymer material, such as polyether ether ketone (PEEK), for instance. In some examples, the core wire 110 can be in the form of solid core. In other examples, the core wire 110 can include a hollow core. In still other examples, the core wire 110 can be formed from multiple strands. In some examples, the core wire 110 can include several combinations of increased and/or decreased profiles along the length of the core wire 110.

In some examples, the guidewire 100 further includes a polymer jacket 120 disposed around the core wire 110. In some examples, the polymer jacket 120 is reflowed over the core wire 110 to fuse the polymer jacket 120 to the core wire 110. In some examples, a layer of the polymer jacket 120 includes a substantially uniform thickness 122, 124 along a length of the core wire 110. In some examples, the layer of substantially uniform thickness 122 is disposed along the distal portion 110A of the core wire 110. In further examples, the layer of substantially uniform thickness 122, 124 is disposed along substantially the entire length of the core wire 110.

In some examples, the first profile 102 of the guidewire 100 (which includes the thickness 122 of the polymer jacket 120 and the diameter or thickness of the core wire 110 at the distal portion 110A of the core wire 110) is smaller than a second profile 104 of the guidewire 100 (which includes the thickness 124 of the polymer jacket 120 and the diameter or thickness of the core wire 110 at the proximal portion 110B of the core wire 110). In some examples, the first profile 102 is used to encompass or describe the size and shape of the distal portion 100A of the guidewire 100, and the second profile 102 is used to encompass or describe the size and shape of the distal portion 100A of the guidewire 100.

In some examples, the core wire 110 of the guidewire 100 can include a tapered portion 110C to transition from the smaller distal portion 110A to the larger proximal portion 110B of the core wire 110. In some examples, the guidewire 100 further includes a coil 130. In some examples, the coil 130 is disposed at the distal portion 110A of the core wire 110. In other examples, the coil 130 is disposed along a portion other than the distal portion 110A of the core wire 110, such as the tapered portion 110C and/or the proximal portion 110B, either in addition to, or instead of, along the distal portion 110A. In some examples, the coil 130 can be attached to the core wire 110. In various examples, the coil 130 can be formed from one or more of various materials, such as, but not limited to, stainless steel, titanium, MP35N, or the like. In some examples, the coil 130 can include materials with radiopaque properties, such as, but not limited to tungsten, platinum, gold, or the like.

In some examples, the coil 130 is completely retained within the polymer jacket 120, such that the coil 130 is disposed completely beneath a surface of the polymer jacket 120. In further examples, the coil 130 is disposed completely within the polymer jacket 120 after the polymer jacket 120 is reflowed. While the example of the guidewire 100 shown in FIG. 1 includes the coil 130, in other examples, it should be understood that the guidewire 100 need not include the coil 130 and, instead includes only the core wire 110 and the polymer jacket 120 at the distal portion 110A of the guidewire 100. Whether the guidewire 100 includes the coil 130 or not can depend on various factors, including the application for which the guidewire 100 is to be used. In some examples, the thickness of the polymer jacket 120 is chosen such that, after reflowing of the polymer jacket 120, the thickness 122 of the polymer jacket 120 is sufficient to completely encapsulate the coil 130, while at the same time maintaining the reduced first profile 102 of the distal portion 100A of the guidewire 100 to allow for access to and navigation within smaller vessels, smaller channels, microchannels, or the like. In some examples, the first profile 102 of the guidewire 100 includes a thickness or diameter of less than 0.014 inches. In further examples, the first profile 102 of the guidewire 100 includes a thickness or diameter of about 0.01 inches. The reduced distal first profile 102 of the guidewire 100, in some examples, includes the polymer jacket 120 of substantially uniform thickness 122 along at least the distal portion 100A of the guidewire 100 formed by reflowing a specially designed polymer material of the polymer jacket 120 over the core wire 110. In some examples, the polymer material of the polymer jacket 120 is suitable for medical use as well as being sufficiently soft to inhibit tissue trauma.

In some examples, the polymer jacket 120 can be made from a flexible polymer material mixed with radiopaque materials or additives in order to enhance visibility of the distal end 100A of the guidewire 100 using various imaging techniques, such as, but not limited to, fluoroscopy. In other examples, the polymer jacket 120 can be made from a flexible polymer material without radiopaque materials if visibility of the polymer jacket 120 of the guidewire 100 is not a concern. In some examples, the polymer jacket 120 can be covered with a coating to impart desired characteristics to the guidewire 100. In further examples, the polymer jacket 120 can be covered with a hydrophilic coating to reduce friction and/or improve torquability of the guidewire 100. In some examples, the polymer jacket 120 can be covered with a hydrophobic coating, if desirable for a particular application. In various examples, the coating can include various materials including, but not limited to, polyvinylpyrrolidone (PVP), hyaluronic acid, or the like. In still other examples, the polymer jacket 120 need not include any coating.

In some examples, the polymer material of the polymer jacket 120 can provide for various properties, such as, but not limited to, hydrophilic properties, hydrophobic properties, or the like, without the need for a coating to achieve such a property. In some examples, this can be achieved through the selection of a particular polymer material and/or with the addition of one or more additives to the polymer material. In some examples, the polymer material of the polymer jacket 120 can include fillers which improve or change physical and mechanical properties of the polymer material, including but not limited to increasing, decreasing, improving, or otherwise changing surface friction, material stiffness, coloring masterbatches, or the like. In some examples, microgranules can be added to the polymer material of the polymer jacket 120, for instance, to increase surface friction of the polymer material. In some examples, the polymer material of the polymer jacket 120 can include fillers which enhance lubricity of the polymer jacket 120.

The guidewire 100, in some examples, can include a tip 140. In some examples, the tip 140 is disposed at the distal end 111 of the core wire 110. In further examples, the tip 140 can be rounded to facilitate atraumatic insertion of the guidewire 100. In some examples, the polymer jacket 120 can be disposed around the tip 140. In other examples, the polymer jacket 120 need not extend around the tip 140, and, instead, can terminate at a location proximal of the tip 140. In some examples, the tip 140 can be an integral part of the core wire 110. In other examples, the tip 140 can be metal or non-metal accessory attached to the core wire 110 or the polymer jacket 120. In some examples, the coil 130 can be attached to the tip 140, either instead of or in addition to the core wire 110, as described above.

With such a configuration, the guidewire 100 includes a decreased distal profile 102 than conventional guidewires, such as the guidewire 200 described herein, to allow the guidewire 100 to navigate through smaller vessels, smaller channels, microchannels, or the like. In some examples, the decreased distal profile 102 of the guidewire 100 allows additional movement of the distal tip 100A, thereby facilitating access to a calcified artery and/or increasing maneuverability of the distal tip 100A of the guidewire 100 to allow maneuvering of an acute bend in vasculature. In some examples, the guidewire 100 can include one or more various other components or devices attached to the guidewire 100, such as a stent, a filter device (for instance, for emboli protection), or the like. In some examples, the guidewire 100 can include multiple combinations of increased and/or decreased profiles of the core wire 110. In some examples, the guidewire 100 may have multiple increments and/or reductions over the length of the guidewire 100 resulting in varied flexibility of the guidewire 100 along the length of the guidewire 100.

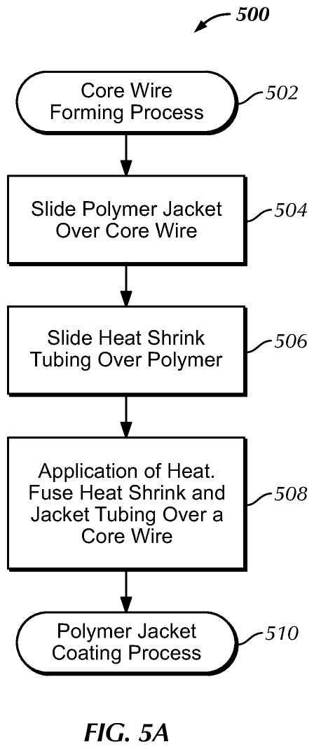

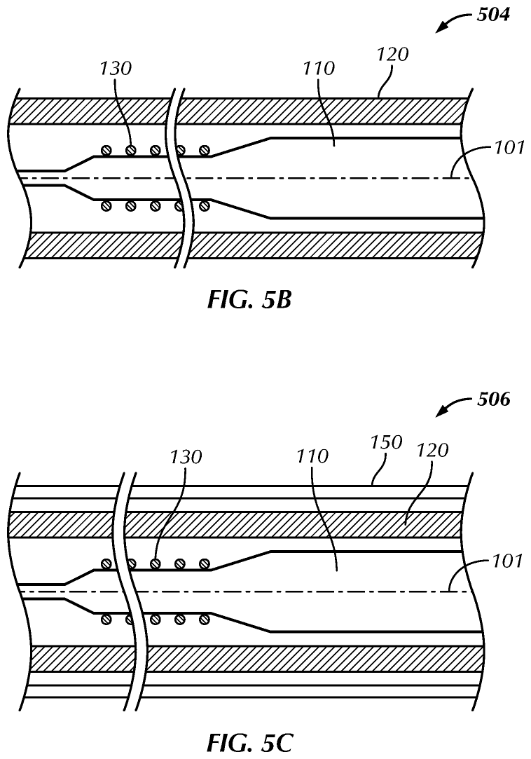

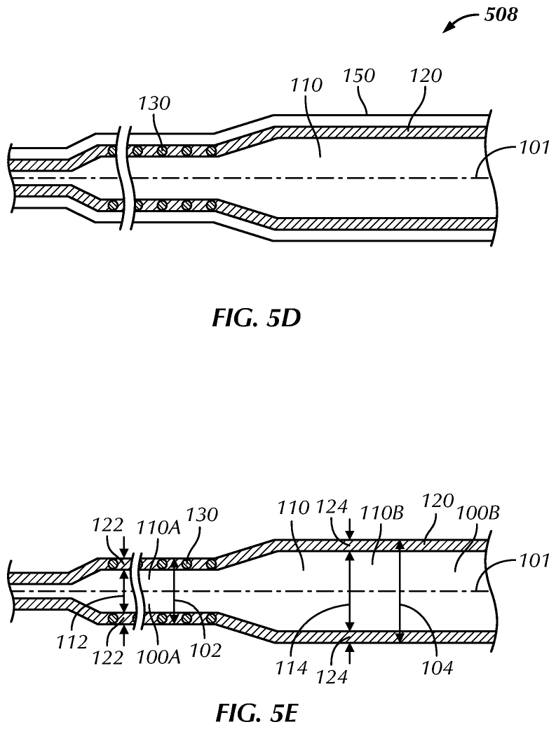

Referring now to FIGS. 5A-5E, a method 500 of making the guidewire 100 is shown. Initially, in some examples, the core wire 110 is formed 502 in the shape, size, and configuration that is desired for the particular application for which the guidewire 100 is to ultimately be used. Once the core wire 110 is formed 502 or otherwise acquired, in some examples, the polymer jacket 120 is placed, slid, or otherwise disposed 504 over the core wire 110. In some examples, the core wire 110 includes the first portion 110A having the first diameter or thickness 112 and the second portion 110B having the second diameter or thickness 114, the first diameter or thickness 112 being smaller than the second diameter or thickness 114.

After the polymer jacket 120 is placed, slid, or otherwise disposed 504 over the core wire 110, in some examples, heat is applied 508 to the polymer jacket 120 and the core wire 110 to reflow the polymer jacket 120 and fuse the polymer jacket 120 to the core wire 110. In some examples, the polymer jacket 120 forms a layer of substantially uniform thickness along a length of the core wire 110.

In some examples, heat shrink tubing 150 is slid or otherwise placed 506 over the polymer jacket 120. In some examples, the heat shrink tubing 150 is slid or otherwise placed 506 over the polymer jacket 120 prior to applying 508 the heat to the polymer jacket 120 and the core wire 110. In this example, the heat shrink tubing 150 acts to constrain the polymer jacket 120 and maintain the positioning and distribution of the polymer jacket 120 around and in proximity to the core wire 110 with application of the heat to the polymer jacket 120 and the core wire 110. Once the polymer jacket 120 is reflowed with the application 508 of heat to the core wire 110, the polymer jacket 120, and the heat shrink tubing 150, the heat shrink tubing 150, in some examples, can be removed from the polymer jacket 120 and the core wire 110. In various examples, the heat shrink tubing 150 can include one or more materials, including, but not limited to, fluorinated ethylene propylene (FEP), polytetrafluoroethylene (PTFE), polyethylene terephthalate (PET), or the like.

In some examples, applying 508 the heat to the polymer jacket 120 and the core wire 110 includes fusing the polymer jacket 120 to the core wire 110 such that the first profile 102 of guidewire 100 (including the polymer jacket 120 and the core wire 110 at the first portion 110A of the core wire 110) is smaller than the second profile 104 of the guidewire 100 (including the polymer jacket 120 and the core wire 110 at the second portion 110B of the core wire 110). In some examples, the first portion 110A of the core wire 110 is proximate the distal end 111 of the core wire 110. In some examples, the second portion 110B of the core wire 110 is disposed between the first portion 110A and the proximal end 113 of the core wire 110. In some examples, the core wire 110 includes the tapered portion 110C disposed between the first portion 110A and the second portion 110B.

In some examples, applying 508 the heat to the polymer jacket 120 and the core wire 110 includes reflowing the polymer jacket 120 to form the layer of substantially uniform thickness along the first portion 110A of the core wire 110. In further examples, the layer of substantially uniform thickness along the first portion 110A of the core wire 110 is achieved without trimming down the polymer jacket 120, as is described above with respect to the guidewire 200. In some examples, the reflow or heat set process attaches the specifically designed polymer material of the polymer jacket 120 over the distally tapered core wire 110 and eliminates the trimming process present in the manufacturing of conventional guidewires, thereby resulting in potential cost and time savings in producing the guidewire 100 as compared to the production of conventional guidewires. In some examples, this results in the polymer jacket 120 closely following the shape of the core wire 110 along its length, thereby resulting in the guidewire 100 including a reduced distal profile 102, as compared to the distal profile of the conventional guidewire. The polymer jacket 120, in some examples, is very thin in order to maintain the reduced distal profile 102. Maintaining such a thin polymer jacket 120 is challenging due to limitations of the tubing extrusion process. Moreover, maintaining such a thin polymer jacket 120 over the core wire 110 requires a stable heat set or reflow process 508.

In some examples, the coil 130 is placed over the core wire 110. In further examples, the coil 130 is disposed completely within the polymer jacket 120 after the polymer jacket 120 is reflowed. That is, in some examples, the polymer jacket 120 completely encapsulates the coil 110. In some examples, the coil 110 is placed over the first portion 110A of the core wire 110. Although shown herein as including the coil, it is not required to include the coil with the guidewire 100. As such, it should be understood that, in some examples, the guidewire 100 can include no coil, for instance, if such a configuration is deemed to be advantageous in a particular procedure.

After the polymer jacket 120 is reflowed and the heat shrink tubing 150 is removed from the polymer jacket 120 and the core wire 110, in some examples, the polymer jacket 120 can then be coated 510. Various coatings are contemplated for the coating process 510, including, but not limited to a hydrophilic coating, a hydrophobic coating, a coating with at least some radiopacity, or the like. In some examples, at least the polymer jacket 120 of the distal portion 100A of the guidewire 100 can be hydrophilically coated, for instance, to improve lubricity by reducing friction. In some examples, the polymer jacket 120 can include a radiopaque element to enhance fluoroscopic imaging of the guidewire 100.

Referring now generally to FIGS. 1 and 3-5E, the present subject matter, in some examples, can be used to create the lower or relatively smaller distal tip profile 102 of the guidewire 100, which, in the present example, can include a polymer-coated guidewire 100. In some examples, this can be achieved while maintaining longitudinal stiffness and force response of the tip 140 of the guidewire 100. In some examples, this present subject matter can allow for the lowered distal tip profile 102 that can facilitate increased free movement of the tip 140 of the guidewire 100. In some examples, the lowered distal tip profile 102 can lead to improved access to calcified vessels with possible microchannels 322 (see FIG. 4). In particular, in some examples, the present subject matter can allow access to microchannels 322 which are not accessible to current straight guidewire profiles, such as the guidewire 200 (which typically have an outer diameter or thickness of 0.014 inches or greater). That is, in some examples, given the lowered distal profile 102 (for instance, having an outer diameter or thickness of about 0.01 inches), the guidewire 100 can access smaller microchannels 322 than could be accessed using a current straight guidewire profile guidewire, such as the guidewire 200. In some examples, the polymer jacket 120 along the distal portion 100A of the guidewire 100 closely follows the shape of the core wire 110, resulting in uniform polymer jacket thickness 122 across the distal portion 100A of guidewire 100.

In some examples, the distally tapered smaller distal profile 102 of the distal portion 100A of the guidewire 100 increases free movement of the distal end 100A of the guidewire 100 and improves the flexibility of the distal portion 100A of the guidewire 100 without reducing torque response. This distally tapered smaller distal profile 102 and/or increased flexibility of the distal portion 100A of the guidewire 100, in some examples, improves access of the guidewire 100 into one or more bends including acute bends of the vasculature and/or a calcified vessel with a possible microchannel. Moreover, in some examples, the thin and uniform polymer jacket 120 allows for multiple re-shaping of the distal portion 100A of the guidewire 100.

Although the core wire 110 shown and described herein includes a particular configuration, in other examples, it should be understood that the configuration of the core wire (including thickness or diameter, shape, lengths of sections, taper, number of tapers, steps, or the like) can be altered to suit different anatomies and/or angles of bends of arteries. Moreover, in some examples, multiple core wires, radiopaque filaments, radiopaque coils, or combinations thereof can be disposed underneath the polymer jacket to suit different anatomies.

The present inventors have recognized various advantages of the subject matter described herein. The present inventors have recognized, among other things, that the present subject matter can be used to provide a guidewire with a relatively small distal tip profile to allow for the guidewire to be maneuvered through smaller vasculature, channels, openings, or the like than conventional guidewires. In various examples, the present subject matter is advantageous in that it provides a guidewire that includes a more flexible distal tip than conventional guidewires to enable, for instance, increased free movement of the distal tip while maintaining longitudinal stiffness and force response of the distal tip. In some examples, the present invention facilitates access to branched vasculature with an acute or otherwise difficult-to-navigate bend. In some examples, the present invention can facilitate the guidewire in crossing/penetrating a lesion with a reduced risk of rupture. While various advantages of the example systems are listed herein, this list is not considered to be complete, as further advantages may become apparent from the description and figures presented herein.

Although the subject matter of the present patent application has been described with reference to various examples, workers skilled in the art will recognize that changes can be made in form and detail without departing from the scope of the subject matter recited in the below claims.

The above Detailed Description includes references to the accompanying drawings, which form a part of the Detailed Description. The drawings show, by way of illustration, specific examples in which the present apparatuses and methods can be practiced. These embodiments are also referred to herein as “examples.”

The above Detailed Description is intended to be illustrative, and not restrictive. For example, the above-described examples (or one or more elements thereof) can be used in combination with each other. Other embodiments can be used, such as by one of ordinary skill in the art upon reviewing the above description. Also, various features or elements can be grouped together to streamline the disclosure. This should not be interpreted as intending that an unclaimed disclosed feature is essential to any claim. Rather, inventive subject matter can lie in less than all features of a particular disclosed embodiment. Thus, the following claims are hereby incorporated into the Detailed Description, with each claim standing on its own as a separate embodiment. The scope of the invention should be determined with reference to the appended claims, along with the full scope of equivalents to which such claims are entitled.

In this document, the terms “a” or “an” are used to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “about” and “approximately” or similar are used to refer to an amount that is nearly, almost, or in the vicinity of being equal to a stated amount.

In the appended claims, the terms “including” and “in which” are used as the plain-English equivalents of the respective terms “comprising” and “wherein.” Also, in the following claims, the terms “including” and “comprising” are open-ended, that is, an apparatus or method that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The Abstract is provided to allow the reader to quickly ascertain the nature of the technical disclosure. It is submitted with the understanding that it will not be used to interpret or limit the scope or meaning of the claims.

1. A method of making a guidewire, the method comprising:
placing a polymer jacket over a core wire, the core wire including a first portion having a first thickness and a second portion having a second thickness, the first thickness being smaller than the second thickness; and
applying heat to the polymer jacket and the core wire to reflow the polymer jacket and fuse the polymer jacket to the core wire, wherein the polymer jacket forms a layer of substantially uniform thickness along a length of the core wire.

2. The method of claim 1, wherein applying the heat to the polymer jacket and the core wire includes fusing the polymer jacket to the core wire such that a first profile of the polymer jacket and the core wire at the first portion of the core wire is smaller than a second profile of the polymer jacket and the core wire at the second portion of the core wire.

3. The method of claim 1, wherein placing the polymer jacket over the core wire includes the first portion of the core wire being proximate a distal end of the core wire.

4. The method of claim 1, wherein placing the polymer jacket over the core wire includes the second portion of the core wire being disposed between the first portion and a proximal end of the core wire.

5. The method of claim 1, comprising:
placing heat shrink tubing over the polymer jacket prior to applying heat to the polymer jacket and the core wire, wherein the heat shrink tubing constrains the polymer jacket with application of the heat to the polymer jacket and the core wire; and
removing the heat shrink tubing from the polymer jacket and the core wire after applying the heat to the polymer jacket and the core wire.

6. The method of claim 1, wherein placing the polymer jacket over the core wire includes the core wire having a tapered portion between the first portion and the second portion.

7. The method of claim 1, comprising placing a coil over the core wire, wherein the coil is disposed completely within the polymer jacket after the polymer jacket is reflowed.

8. The method of claim 7, wherein placing the coil over the core wire includes placing the coil over the first portion of the core wire.

9. The method of claim 1, wherein applying heat to the polymer jacket and the core wire includes reflowing the polymer jacket to form the layer of substantially uniform thickness along the first portion of the core wire.

10. The method of claim 9, wherein applying heat to the polymer jacket and the core wire includes achieving the layer of substantially uniform thickness along the first portion of the core wire without trimming down the polymer jacket.

11. A guidewire comprising:
a core wire including a first portion having a first thickness and a second portion having a second thickness, the first thickness being smaller than the second thickness; and
a polymer jacket reflowed over the core wire to fuse the polymer jacket to the core wire, wherein a layer of the polymer jacket includes a substantially uniform thickness along a length of the core wire.

12. The guidewire of claim 11, wherein the first portion of the core wire is proximate a distal end of the core wire.

13. The guidewire of claim 11, wherein the second portion of the core wire is disposed between the first portion and a proximal end of the core wire.

14. The guidewire of claim 11, wherein a first profile of the polymer jacket and the core wire at the first portion of the core wire is smaller than a second profile of the polymer jacket and the core wire at the second portion of the core wire.

15. The guidewire of claim 11, wherein the core wire includes a tapered portion between the first portion and the second portion.

16. The guidewire of claim 11, comprising a coil disposed over the core wire, the coil being disposed completely within the polymer jacket after the polymer jacket is reflowed.

17. The guidewire of claim 16, wherein the coil is disposed over the first portion of the core wire.

18. The guidewire of claim 11, wherein the polymer jacket is reflowed to form the layer of substantially uniform thickness along the first portion of the core wire.

19. The guidewire of claim 11, wherein the first profile of the guidewire includes a thickness of less than 0.014 inches.

20. The guidewire of claim 11, wherein the first profile of the guidewire includes a thickness of about 0.01 inches.
